(19) United States
(12) Patent Application Publication
(10) Pub. No.: US 2023/0056572 A1
(43) Pub. Date: Feb. 23, 2023

(54) EYE DROP DISPENSER ASSEMBLY

(71) Applicant: Frank G. Burnett, Los Angeles, CA (US)

(72) Inventor: Frank G. Burnett, Los Angeles, CA (US)

(21) Appl. No.: 17/571,693

(22) Filed: Jan. 10, 2022

Related U.S. Application Data
(60) Provisional application No. 63/235,427, filed on Aug. 20, 2021.

(51) Int. Cl. A61F 9/00

(52) U.S. Cl. CPC A61F 9/0026

(57) ABSTRACT

An eye drop dispenser assembly which covers and comfortably encloses the user's eyes and dispenses medicine in a manner which obviates blinking. The dispenser assembly comprises two portions, each having an eye drop dispensing port; an eye drop reservoir; an eye drop dispensing button; a fluid connection between the eye drop reservoir and the eye drop dispensing port via the eye drop dispensing button; and a cushion positioned to surround a user's eye when the eye drop dispenser assembly is in place on a user, and a hinge assembly for connecting the two portions so that the two portions can move relative to each other to assume a curved configuration that places each portion over the user's eye whereby eye drops are dispensed directly into the user's eye and for folding the two portions in a compact relationship for easy storage.

### CROSS REFERENCE TO RELATED APPLICATIONS

This application claims priority to U.S. Provisional Patent Application Ser. No. 63/235,427 filed Aug. 20, 2021, the disclosure of which is incorporated herein by reference in its entirety.

### BACKGROUND OF THE INVENTION

### 1. Field of the Invention

The present invention relates, generally, to devices and methods for delivery of medication and improving the rate of successful medication adherence. More particularly, the present invention relates to improving the rate of successful delivery of medication in the form of eye drops from an eye drop bottle to an eye.

### 2. Brief Description of the Prior Art

A number of medical conditions, such as, for example, glaucoma, conjunctivitis, and allergies may be treated by the use of eye drops. Eye drops may contain steroids, antihistamines, antibiotics. prostaglandin analogs, beta blockers, alpha agonists, carbonic anhydrase inhibitors, or other drugs. Additionally, eye drops may be used to lubricate the eye or replace tears. In order for the treatment with eye drops to be effective, good adherence to treatment, i.e., remembering to use the eye drop and getting sufficient amount of the eye drop into the eye, is needed. Some adherence studies of glaucoma patients indicate that approximately 45% of patients demonstrate poor ability to remember to use their eye drops, and other studies show that when patients do attempt to administer eye drops, they frequently miss the eye.

One reason for poor adherence is the inability to successfully administer an eye drop. Patients may accidentally miss the eye instead placing the drop on the eyelids or cheek, or they may touch the bottle tip against the ocular surface causing contamination of the bottle and possible ocular surface damage. In a recent study, over 30% of patients missed the eye while attempting to instill an eye drop, and over 75% touched the bottle tip to the ocular surface or eyelids. Only 10% of patients exhibited proper technique. See for example, Gupta R, Patil B, Shaw B M, “Evaluating eye drop instillation technique in glaucoma patients,” Journal of Glaucoma, 2012, 21: 189-92. Correctly placing an eye drop through conventional means tends to be challenging.

Researchers and eye care providers have had difficulty developing a suitable device to improve the success rate of instilling eye drops. Existing eye drop delivery devices have not been widely adopted due to their difficulty of use, intimidating nature, and lack of efficacy.

Most people encounter difficulty in applying drops to their eyes due to the extreme sensitivity of them. The eye is sensitive. Thus, normally individuals find it difficult to control reflexive blinking when applying eye drops. Moreover, poor vision makes it difficult to properly position or align an eye dropper bottle relative to the eye frequently causes drops to be improperly applied.

Thus, applying eye drops is generally difficult, uncomfortable and resulted in little, if any, of each drop entering the eye so that several attempts were necessary to insure placement of the ophthalmic solution in the eye. As a result, considerable amounts of the solution were wasted with no real assurance that a prescribed or desired amount of solution was placed in the eye.

It is already part of prior art practice employing commercially available squeeze bottle or equivalent eye drop dispensers to use positioning devices to direct the dispensed eye drops onto the eye surface where needed. Such positioning devices are exemplified by the tripod support of Campagna, et al. of U.S. Pat. No. 3,934,590, issued on Jan. 27, 1976, and the eye-encircling dispenser support of Bechtle of U.S. Pat. No. 4,531,944, issued on Jul. 30, 1985. These, and all other known prior art combined supports and dispensers, while beneficial, still have shortcomings. Such shortcomings include requiring special head positions and other special procedures or techniques that, at best, make self-application difficult and, more often, prevent the user from achieving proper eye treatment using eye drops. The prior art dispensers can sometimes even contribute to the spreading of the eye infection with an inadvertently contaminated dispenser.

There is, therefore, a need in the art to provide an eye drop dispenser assembly which covers and comfortably encloses the user's eyes and which dispenses medicine in a manner which obviates blinking.

There is a further need in the art to provide an eye drop dispenser assembly which contains and safely dispense medicine into the eyes of a user.

### SUMMARY OF THE INVENTION

The present invention meets these needs. More particularly, the present invention provides an eye drop dispenser assembly which covers and comfortably encloses the user's eyes and dispenses medicine in a manner which obviates blinking. The eye drop dispenser assembly comprises means for containing and safely dispensing the medicine. Additionally, the eye drop dispenser assembly of the invention is constructed and arranged such that it can fold in half to prevent dirt and other contaminates from entering the dispensing ports while also giving the user a compact design for easy carrying and storage which allows it to fit in the user's pocket or purse. That is, a hinge assembly connects the first portion to a second portion.

The present invention provides an eye drop dispenser assembly, comprising: a first portion which has a housing with a first wall which is an inner wall that abuts a user's face adjacent to the user's eyes when the eye drop dispenser assembly is in place on the user; a second wall which is a front wall when the eye drop dispenser assembly is in place on the user; and a third wall which is a top wall when the eye drop dispenser assembly is in place on the user; a nose-engaging element on the first wall that abuts the user's nose when the eye drop dispenser assembly is in place on the user; and a cushion on the first wall positioned to surround the user's eye that is associated with the first portion when the eye drop dispenser assembly is in place on the user.

The first portion further includes: a fluid reservoir; a fluid inlet fluidically connected to the fluid reservoir; a closure element on the fluid inlet; an eye-drop dispensing port on the first wall in a location that is positioned immediately adjacent to the user's eye that is associated with the first portion when the eye drop dispenser assembly is in place on the user; an eye-drop dispensing control button on the first portion; a fluid connection between the fluid reservoir and the eye-drop dispensing port via the control button, and a hinge assembly for hingeably connecting the first portion to a second portion which is identical to the first portion.

The hinge assembly is structured such that the first portion can move relative to the second portion and so that the first and second portions can assume a configuration that places each portion of the first and second portions over the user's eye that is associated with that portion with the cushion of the first portion snugly abutting the user's face around the user's eye associated with the first portion and the eye-drop dispensing port of the first portion positioned adjacent to the user's eye that is associated with the first portion whereby eye drops are dispensed with increased precision from the adjustable dispensing ports directly into the user's eye. Also, the hinge assembly of the eye drop dispenser assembly of the invention is constructed and arranged such that the first portion and the second portion can fold in half to prevent dirt and other contaminates from entering the dispensing ports while also giving the user a compact design for easy carrying and storage which allows the eye drop dispenser assembly to tit in the user's pocket or purse.

The present invention is intended to provide a safe and effective eye drop dispensing method and components for its practice, overcoming the foregoing and other shortcomings of the prior art. Using the present invention, eye drops are more efficiently administered, reducing waste. This is of particular importance with certain medicinal eye drops, which are particularly expensive. As such, the present invention may be used even by the elderly with shaking hands or by patients with poor eyesight, crippling arthritis or other heretofore interfering handicaps.

These and other features and advantages of the present invention will be better appreciated and understood when the following description is read in light of the accompanying drawings.

### BRIEF DESCRIPTION OF THE DRAWINGS

The components in the figures are not necessarily to scale, emphasis instead being placed upon illustrating the principles of the invention. Moreover, in the figures, like referenced numerals designate corresponding parts throughout the different views.

FIG. 1 is a front perspective view of an eye drop dispenser assembly of the present invention.

FIG. 2 is a rear perspective view of the eye drop dispenser assembly of FIG. 1.

FIG. 3 is a schematic diagram of a fluid circuit contained in the eye drop dispenser assembly of the invention.

FIG. 4 is a top perspective view of the eye drop dispenser assembly of the invention in a curved configuration for use on the face and over the eyes of a user.

### DETAILED DESCRIPTION OF THE INVENTION

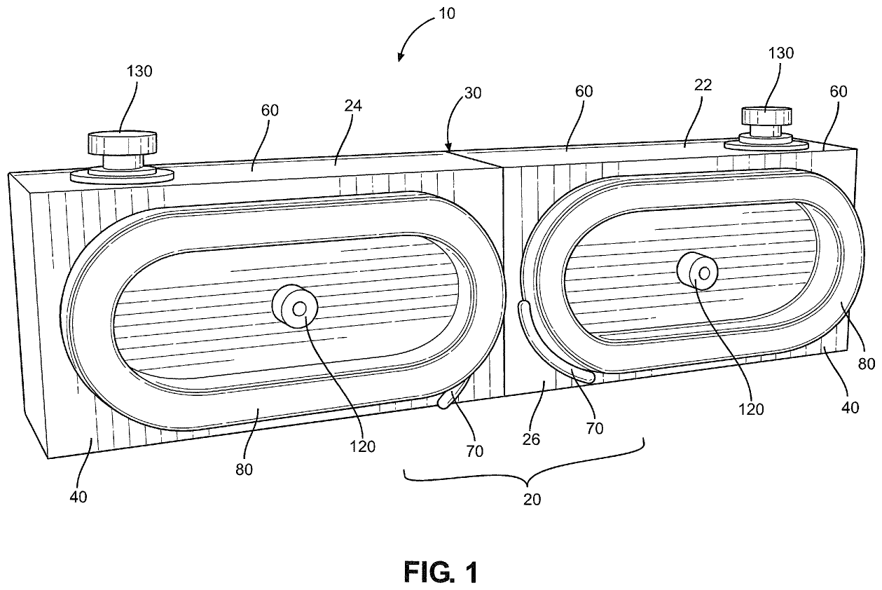

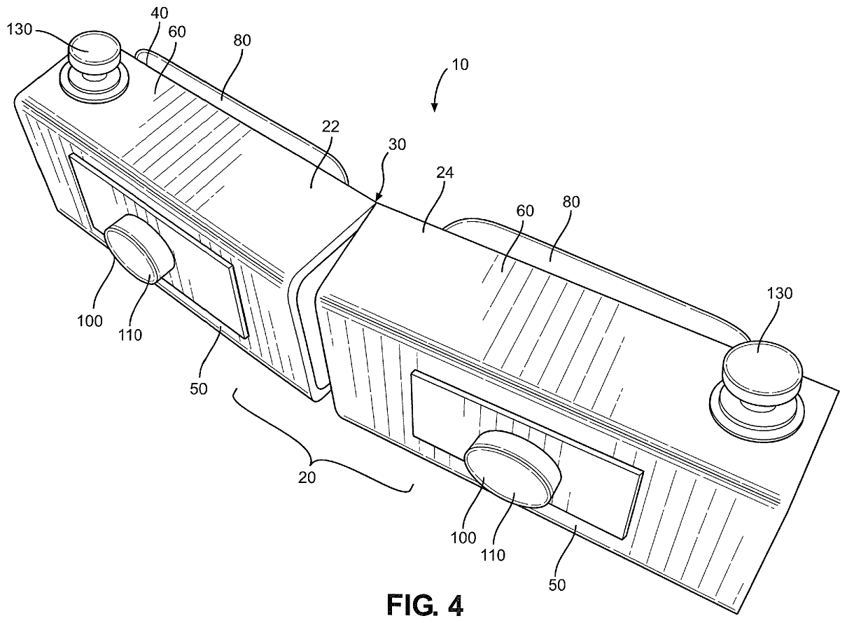

Referring to FIGS. 1 through 4, the present invention illustrates an eye drop dispenser assembly 10. In use, eye drop dispenser assembly 10 is placed over the user's eyes and the user dispenses eye drops directly into his or her eyes. Eye drop dispenser assembly 10 comprises a container 20 with two identical portions 22 and 24, which abut each other and which are hingeably connected together by a hinge assembly 30 such that container 20 can flex at a central location to move one portion relative to the other portion so that the eye drop dispenser assembly 10 forms a curved configuration as shown in FIG. 4 so as to conform to the curve of the user's face in the vicinity adjacent to the user's eyes in a manner so as to be comfortably and securely placed over the user's eyes.

As disclosed herein above, container 20 comprises identical portions 22 and 24. Since these portions 22 and 24 are identical, only portion 22 will be described herein below, it being understood that the description of portion 22 also applies to portion 24.

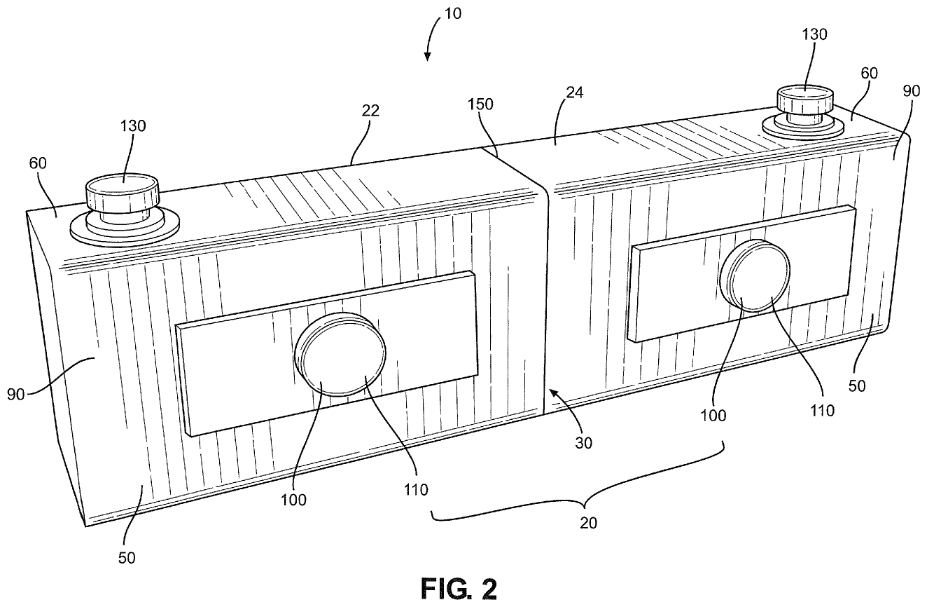

Referring particularly to FIGS. 1 and 2, first portion 22 is in the form of a housing which comprises: a first wall 40 which is an inner wall which abuts the user's face adjacent to the user's eyes when eye drop dispenser assembly 10 is positioned relative to the user's face; a second wall 50 which is an outer wall when eye drop dispenser assembly 10 is positioned relative to the user's face; and a third wall 60 which is a top wall when the eye drop dispenser assembly 10 is in place on the user. First portion 22 further comprises a nose-engaging element 70 on the first wall 40 which abuts the user's nose when eye drop dispenser assembly 10 is in place relative to the user's face; and a face engaging light blocking cushion 80 on first wall 40. Face engaging light blocking cushion 80, which is positioned such as to surround the user's eye which is associated with first portion 22 when dispenser assembly 20 is in place on the user's face. Cushion 80 acts to comfortably seat dispenser assembly 10 on the user and it also acts to block the light from reaching the user's eye. If light is being blocked from the user's eye, the user most likely will not to see any eye drops being directed into his eye, and therefore, is not likely to blink. That is, blinking may waste fluid, which may frustrate the user.

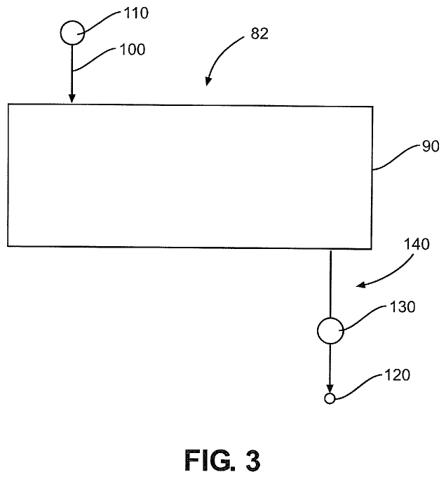

Referring to FIGS. 1 through 4, first portion 22 and second portion 24 further comprise an eye-drop dispensing system 82 (FIG. 3). Eye-drop dispensing system 82 for both portions 22, 24 is identical; therefore, only the eye-drop dispensing system 82 for first portion 22 will be discussed herein below.

With particular reference to FIG. 3, eye drop dispensing system 82 comprises: a fluid reservoir 90; a fluid inlet 100 which is fluidically connected to fluid reservoir 90; a closure element 110 on fluid inlet 100; and an eye drop dispensing port 120, which as particularly shown in FIG. 1, is located on first wall 40 of portions 22, 24 in a location that is positioned immediately adjacent to the user's eye which is associated with first portion 22 and second portion 24 when eye drop dispenser assembly 10 is in place on the user. FIGS. 2 and 4 further illustrate fluid inlet 100 and closure element 110 on fluid inlet 100. As shown in FIG. 1, eye drop dispensing port 120 is located centrally of light-blocking cushion 80 such that the eye drops will be dispensed from dispensing port 120 and directly into the user's eye. Referring particularly to FIG. 3, eye drop dispensing system 82 further comprises an eye drop dispensing control button 130 located on top wall 60 of portions 22, 24, and a fluid connection 140 between fluid reservoir 90 and eye drop dispensing port 120 via control button 130.

Referring particularly to FIG. 2, each portion 22, 24 further comprises a hinge assembly element 150. It is to be appreciated that both hinge assembly elements 150 are constructed and arranged to form hinge assembly 30. As disclosed herein above, hinge assembly 30 connects first portion 22 to second portion 24 so that first portion 22 and second portion 24 can move relative each other to assume a curved configuration as shown in FIG. 4. It is to be appreciated, that the curved configuration of FIG. 4 enables each portion 22, 24 of eye drop dispenser assembly 10 to be directly positioned over each eye of the user with cushions 80 snugly positioned against the user's face, and particularly, around the user's eyes, and with the eye-drop dispensing port 120 of each portion 22, 24 being positioned adjacent to the user's eyes, whereby eye drops are safely and conveniently dispensed from dispending port 120 of each portion 22, 24 and directly into the eyes of the user.

It is to be understood and appreciated that eye drop dispenser assembly 10 of the invention may have an eye drop dispensing system 82 in both portions 22, 24, or it may have only eye drop dispensing system for both portions 22, 24, without departing from the scope of the invention. In FIG. 2, the reference numeral 90 appears associated with each portions 22, 24 to indicate that the fluid reservoir 90 of the eye drop dispensing system 82 is housed in portions 22 and 24.

Once fluid inlet 100 and dispensing control button 130 are closed, the eye-drop dispensing system 82 is closed. Operation of button 130 forces fluid out of fluid reservoir 90 and out through eye drop dispensing port 120. This function can be carried out by pressure within the eye drop fluid dispensing system 82, which is created by the closed nature of the system by forcing button 130 to operate in a manner that increases pressure within the system toward the eye drop dispensing port 120, or by applying an external pressure source to the system through the fluid inlet 100.

The eye drop dispenser assembly 10 is used by filling the fluid reservoir 90 with suitable eye medicine; placing the eye drop dispenser assembly 10 on the user's face covering his eyes with the cushions 80 surrounding the user's eyes to ensure comfortable and secure positioning of the eye drop dispenser assembly 10, as well as blocking and preventing light from reaching the user's eyes. The eye medicine is then dispensed directly into the user's eyes by operating control button 130 of both portions 22, 24.

Also, as disclosed herein above, the hinge assembly 30 of the eye drop dispenser assembly 10 of the invention is constructed and arranged such that the first portion 22 and the second portion 24 of container 20 can be folded together and against each other, thereby preventing dirt and other contaminates from entering the dispensing ports 120, while also giving the user a compact design for easy carrying and storage of the eye drop dispenser assembly 10 which allows the eye drop dispenser assembly 10 to easily and conveniently fit into the user's pocket or purse.

While the present invention has been described in connection with the preferred embodiments of the figures, it is to be understood that other similar embodiments may be used or modifications and additions may be made to the described embodiments for performing the same function of the present invention without deviating therefrom. Accordingly, it is intended by the appended claims to cover all such changes and modifications as come within the scope and spirit of the invention.

What is claimed is:

1. An eye drop dispenser assembly, comprising:
a first portion and a second portion, each portion comprising:
a housing having a first wall which is an inner wall that abuts a user's face adjacent to the user's eyes when the eye drop dispenser assembly is in place on the user; a second wall which is an outer wall when the eye drop dispenser assembly is in place on the user; and a third wall which is a top wall when the eye drop dispenser assembly is in place on the user; a nose-engaging element on the first wall which abuts the user's nose when the eye drop dispenser assembly is in place on the user; and a cushion on the first wall positioned to surround the user's eye that is associated with the first portion when the eye drop dispenser assembly is in place on the user;
an eye drop dispensing system associated with the first portion and the second portion; and,
a hinge assembly for connecting the first portion and the second portion together such that the first portion and the second portion can move relative to each other so the first portion and the second portion assume a configuration that places each portion of the first and second portions over the user's eye that is associated with that portion with the cushion of the first portion snugly abutting the user's face around the user's eye associated with the first portion and the eye-drop dispensing port of the first portion positioned adjacent to the user's eye that is associated with the first portion whereby eye drops are dispensed from the dispending port directly into the user's eye.

2. The eye drop dispenser assembly of claim 1, wherein the eye drop dispensing system comprises:
a fluid reservoir,
a fluid inlet fluidically connected to the fluid reservoir,
a closure element on the fluid inlet,
an eye-drop dispensing port on the first wall of the first portion and the second portion in a location that is positioned immediately adjacent to the user's eye that is associated with the first portion when the eye drop dispenser assembly is in place on the user;
an eye-drop dispensing control button on the first portion; and
a fluid connection between the fluid reservoir and the eye-drop dispensing port via the control button.

3. The eye drop dispenser assembly of claim 1, wherein the first and second portions are identical.

4. The eye drop dispenser assembly of claim 1, wherein the hinge assembly is constructed and arranged to allow the first portion and the second portion to be folded against each other in a compact relationship for easy storage of the eye drop dispenser assembly.

5. An eye drop dispenser assembly, comprising:
at least two portions, each portion comprising:
an eye drop dispensing port,
an eye drop reservoir,
an eye drop dispensing button,
a fluid connection between the eye drop reservoir and the eye drop dispensing port via the eye drop dispensing button,
a cushion positioned to surround a user's eye when the eye drop dispenser assembly is in place on a user, and
a hinge assembly for connecting the two portions together so the two portions can move relative to each other and assume a configuration that places each portion of the first and second portions over the user's eye that is associated with that portion with the cushion of the each portion snugly abutting the user's face around the user's eye associated with that portion and the eye drop dispensing port is positioned adjacent to the user's eye that is associated with that portion whereby eye drops are dispensed from the dispensing port directly into the user's eye.

6. The eye drop dispenser assembly of claim 5, wherein each portion further comprises a cushion structured and positioned to surround the user's eye that is associated with each portion when the eye drop dispenser assembly is in place on the user.

7. The eye drop dispenser assembly of claim 5, wherein the hinge assembly is constructed and arranged to allow the two portions to be folded against each other in a compact relationship for easy storage of the eye drop dispenser assembly.
